(19) United States
(12) Patent Application Publication
(10) Pub. No.: US 2017/0065661 A1
(43) Pub. Date: Mar. 9, 2017

(54) CHINESE MEDICINE PRESCRIPTION CAPABLE OF TREATING LUNG ADENOCARCINOMA

(71) Applicant: KUAN-CHIH CHOW, Nantou County (TW)

(72) Inventor: KUAN-CHIH CHOW, Nantou County (TW)

(21) Appl. No.: 14/849,548

(22) Filed: Sep. 9, 2015

(51) Int. Cl. A61K 36/9068; A61K 36/284; A61K 36/484; A61K 36/07; A61K 36/725; A61K 36/28; A61K 36/48; A61K 36/232

(52) U.S. Cl. CPC A61K 36/9068; A61K 36/48; A61K 36/284; A61K 36/232; A61K 36/07; A61K 36/725; A61K 36/28; A61K 36/484; A61K 2236/331

(57) ABSTRACT

A Chinese medicine prescription capable of treating lung adenocarcinoma is composed of Polygala tenuifolia Willd, atractylodes macrocephala, angelica sinensis, poria pararadicis, astragalus membranaceus, ziziphus jujaba Mill.var.spinosa, codonopsis pilosula, costustoot, radix glycyrrhizae preparata and fresh ginger, and a pharmaceutical residue is filtered and obtained after a water quenching procedure of the aforementioned herbs to provide a water-quenched medicine soup, and a pharmaceutical residue is filtered and obtained after processing the water-quenched pharmaceutical residue by a liquor quenching procedure to obtain a liquor-quenched medicine soup; and the water-quenched medicine soup and the liquor-quenched medicine soup are mixed. The prescription is capable of triggering cell autophagy and reducing cell growth, expansion and metastasis, so as to achieve the effect of lung adenocarcinoma treatment.

### FIELD OF THE INVENTION

The technical field relates to a Chinese medicine prescription capable of treating lung adenocarcinoma, and more particularly to the Chinese medicine prescription with the effects of causing lung adenocarcinoma cell autophagy and reducing cell growth, expansion and metastasis for treating lung adenocarcinoma.

### BACKGROUND OF THE INVENTION

Cancer is one of the top ten causes of death and has become the leading cause of death for 33 consecutive years. The main factor causing cancer is that cells become abnormal and keep dividing to form more abnormal cells automatically. At present, Western medicine therapy such as surgery, radiation therapy, chemotherapy, hormone therapy, or biopharmaceutical therapy used for cancer treatment often causes serious sides effects on a patient's body and even has cases of cancer cell expansion and metastasis to other organs. Therefore, it is a blessing to cancer patients to have a mild treatment capable of inhibiting the growth, expansion and metastasis of cancer cells.

Autophagy is a method for cells to maintain homeostasis, and autophagy was first discovered in yeast. At present, 27 types of specific genes are identified and universally named as autophagy-related genes (atg).

Cell autophagy is mainly divided into four major types: macroautophagy, microautophagy, chaperone-mediated autophagy (CMA) and mitophagy, wherein the mitophagy is often considered as a part of macroautophagy; the mitophagy includes an ATPase family, AAA domain containing 3A (ATAD3A) adenosine triphosphatase and has a molecular weight of 67-kDa (kilodaltons), and exists at the positions of endoplasmic reticulum, mitochondria-associated membrane (MAM), transport vesicles and mitochondrial outer membrane, and it is a member of ATAD3 protein family.

In addition, dihydrodiol dehydrogenase (DDH) also known as aldo-keto reductase 1C2 (AKR1C2) having high expression in lung adenocarcinoma cells has a molecular weight of 36-kDa and a capability of quickly converting anticancer chemicals. In addition, the DDH will also manufacture DNA repair-related proteins and enzymes, so that the high expression of the DDH will increase the drug resistance of cancer cells against anticancer chemicals and radiation therapy.

### SUMMARY OF THE INVENTION

Therefore, it is a primary objective of this disclosure to provide a Chinese medicine prescription capable of treating lung adenocarcinoma.

To achieve the aforementioned objective, this disclosure provides a Chinese medicine prescription composed of Polygala tenuifolia Willd, atractylodes macrocephala, angelica sinensis, poria pararadicis, astragalus membranaceus, ziziphus jujaba Mill.var.spinosa, codonopsis pilosula, costustoot, radix glycyrrhizae preparata, fresh ginger, wherein the Chinese medicine prescription capable of treating lung adenocarcinoma triggers lung adenocarcinoma cell autophagy and reduces cancer cell nest growth, expansion and metastasis.

In the aforementioned Chinese medicine prescription capable of treating lung adenocarcinoma, Polygala tenuifolia Willd, atractylodes macrocephala, fresh ginger, costustoot and codonopsis pilosula are major regulators for inhibiting lung adenocarcinoma cell growth, expansion and metastasis.

The aforementioned Chinese medicine prescription is composed of 6 parts by weight of Polygala tenuifolia Willd, 3 parts by weight of atractylodes macrocephala, 3 parts by weight of angelica sinensis, 3 parts by weight of poria pararadicis, 3 parts by weight of astragalus membranaceus, 3 parts by weight of ziziphus jujaba Mill.var.spinosa, 6 parts by weight of codonopsis pilosula, 3 parts by weight of costustoot, 1.5 parts by weight of radix glycyrrhizae preparata, and 1.5 parts by weight of fresh ginger. The Chinese medicine prescription is capable of triggering lung adenocarcinoma cell autophagy, reducing cancer cell nest growth, expansion and metastasis, so as to achieve the effect of cancer treatment.

In the aforementioned composition of the Chinese medicine prescription, the composition of the Chinese medicine prescription includes a pharmaceutical residue filtered and obtained after a water quenching procedure takes place, so as to obtain a water-quenched medicine soup; and a pharmaceutical residue filtered and obtained after processing the water-quenched pharmaceutical residue by a liquor quenching procedure to obtain a liquor-quenched medicine soup; and the water-quenched medicine soup and the liquor-quenched medicine soup are mixed.

In the aforementioned composition of the Chinese medicine prescription, the water quenching procedure extracts the herbs by 100 g of water at 60° C. for 20 minutes; and the liquor quenching procedure extracts the herbs by 100 g of 50˜90% alcohol at 60° C. for 15 minutes.

This disclosure is definitely useful and inventive and worthy for being promoted in related industries and disclosed to the public.

### BRIEF DESCRIPTION OF THE DRAWINGS

FIG. 1 is an electropherogram of a two-dimensional electrophoresis in accordance with this disclosure;

FIG. 2 is a schematic view showing the mechanism and procedure of transporting proteins to a new path of mitochondria in accordance with this disclosure;

FIG. 3 is a microscopic image of silencing DRP1 in accordance with this disclosure;

FIG. 4 is a microscopic image of silencing ATATA3 in accordance with this disclosure;

FIG. 5 is a microscopic image of a cell autophagy in accordance with this disclosure;

FIG. 6 is an electropherogram of screening cancer cells in accordance with this disclosure;

FIG. 7 is an inverted microscopic view of screening stromal cells and enthothelial cells in accordance with this disclosure;

FIG. 8 is a computed tomogram of a lung adenocarcinoma patient having an eight-week treatment in accordance with this disclosure, and

FIG. 9 is a cross-sectional view of a computed tomogram of a lung adenocarcinoma patient having an eight-week treatment in accordance with this disclosure.

### DESCRIPTION OF THE PREFERRED EMBODIMENTS

The aforementioned and other objectives and advantages of this disclosure will become clearer in light of the following detailed description of an illustrative embodiment of this invention described in connection with the drawings. The embodiment briefly describes the cause of triggering the cell autophagy and related experiment data for supporting the advantages and effects of this disclosure.

In an embodiment of this disclosure, the Chinese medicine prescription capable of treating lung adenocarcinoma includes 6 parts by weight of Polygala tenuifolia Willd, 3 parts by weight of atractylodes macrocephala, 3 parts by weight of angelica sinensis, 3 parts by weight of poria pararadicis, 3 parts by weight of astragalus membranaceus, 3 parts by weight of ziziphus jujaba Mill.var.spinosa, 6 parts by weight of codonopsis pilosula, 3 parts by weight of costustoot, 1.5 parts by weight of radix glycyrrhizae preparata, and 1.5 parts by weight of fresh ginger. A pharmaceutical residue is filtered and obtained after a water quenching procedure takes place for the aforementioned herbs in their respective parts by weight, so as to obtain a water-quenched medicine soup; and a pharmaceutical residue is filtered and obtained after processing the water-quenched pharmaceutical residue by a liquor quenching procedure to obtain a liquor-quenched medicine soup; and the water-quenched medicine soup and the liquor-quenched medicine soup are mixed. The water quenching procedure extracts the herbs by 100 g of water at 60° C. for 20 minutes; and the liquor quenching procedure extracts the herbs by 200 g of 50˜90% alcohol at 60° C. for 15 minutes.

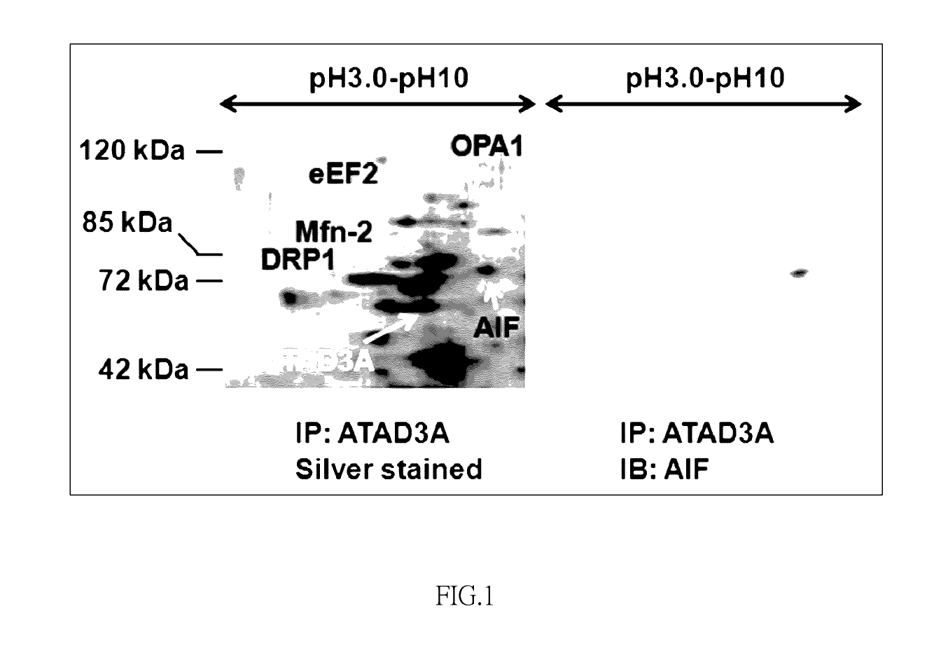

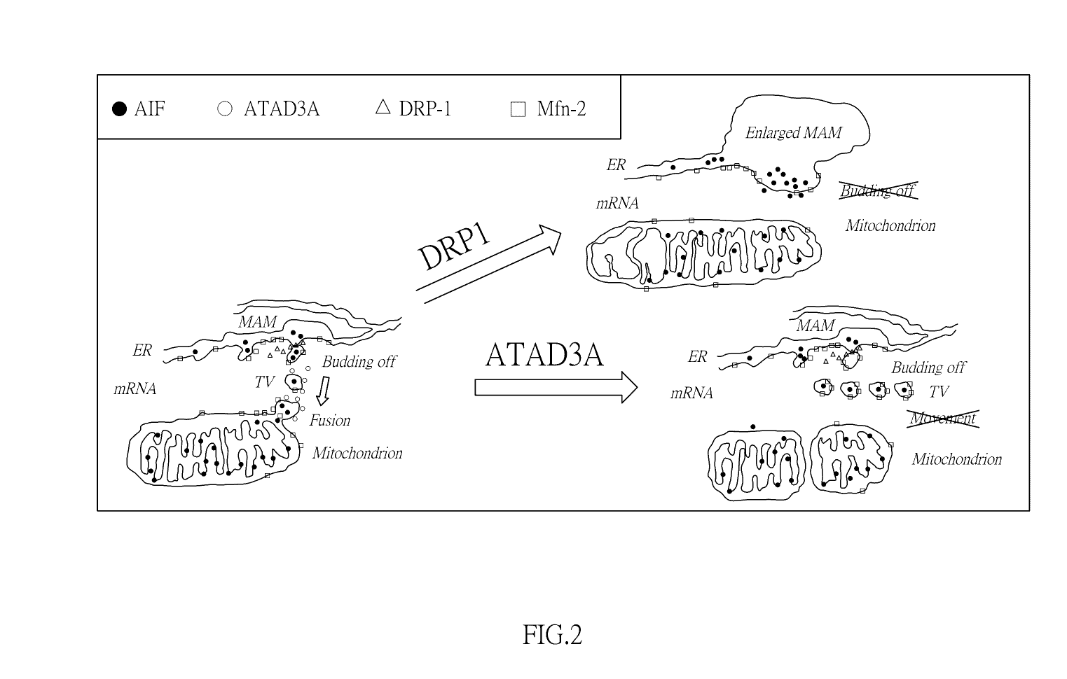

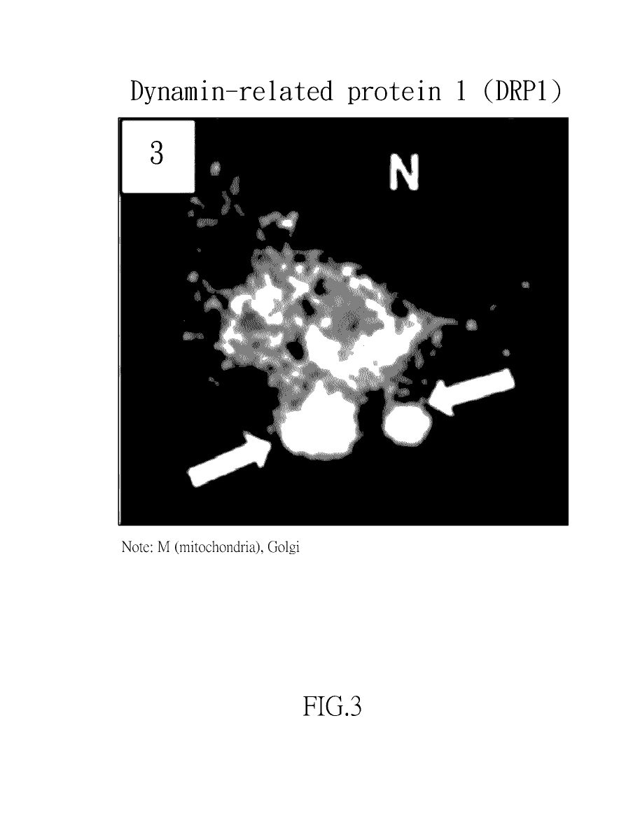

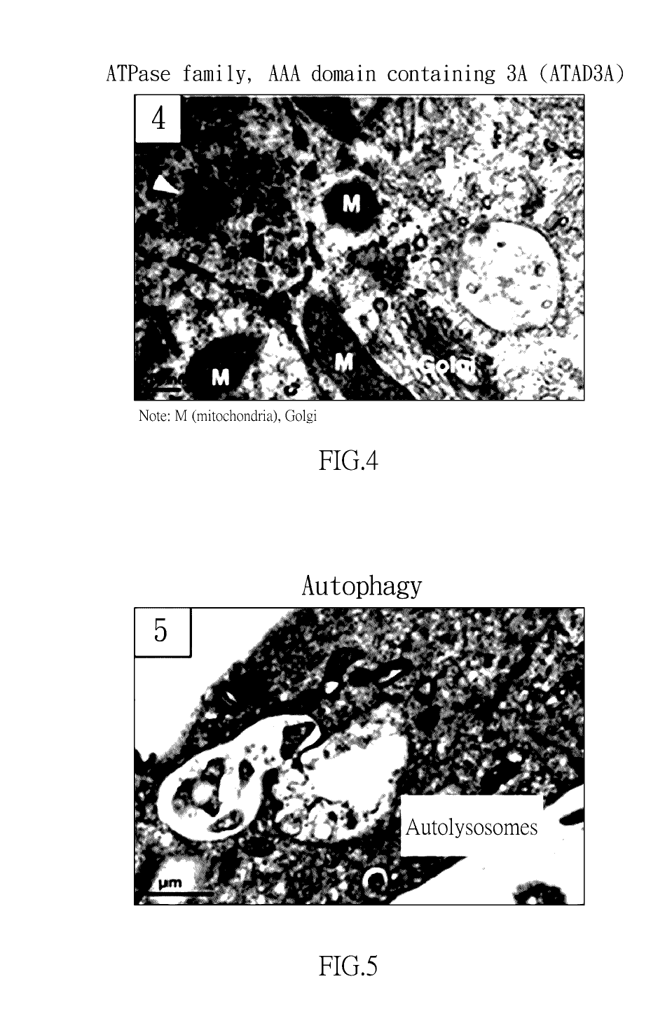

In this disclosure, if the ATAD3A monoclonal antibody is used for the immunoprecipitation of the light membrane and mitochondria-associated membrane (MAM) of cells as shown in FIG. 1, the glucose gradient ultracentrifugation uses a two dimensional electropherogram to analyze mitofusin 2 (Mfn-2), optic atrophy 1 (OPA1), dynamin-related protein 1 (DRP 1), eukaryotic elongation factor-2 (eEF2) and apoptosis-inducing factor (AIF) as indicated by the white arrow in FIG. 1. According to the aforementioned result, this is a new path of transporting proteins to mitochondria. With reference to FIG. 2 for the mechanism of the aforementioned new path, the proteins required by the cells are manufactured in endoplasmic reticulum (ER), and mitochondria-associated membrane (MAM) and transport vesicle (TV) are fused into mitochondria (mitochondrion), wherein when the DRP 1 is silenced as shown in FIG. 3, the cytoplasm produces enlarged MAM and AIF and causes budding off, and thus affecting the manufacture of proteins. In FIG. 4, the silenced ATAD3A will affect the interrupt of the movement after the endoplasmic reticulum (ER) manufactures the proteins. The foregoing two enzymes are the necessary factors of the new path. In FIG. 5, a deficiency of cell autophagy produces autolysosomes.

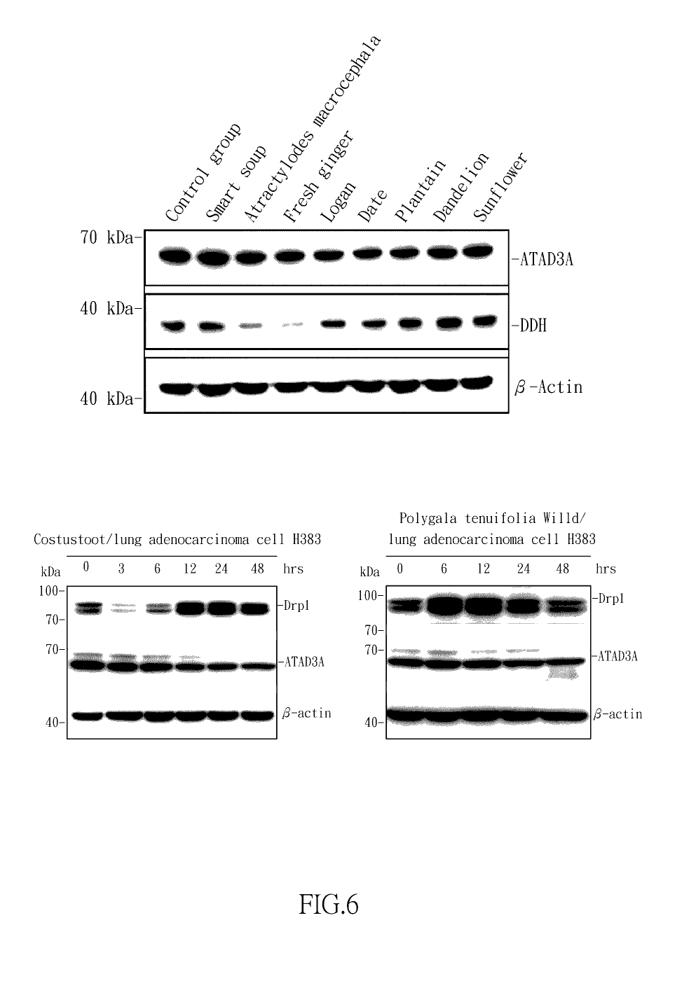

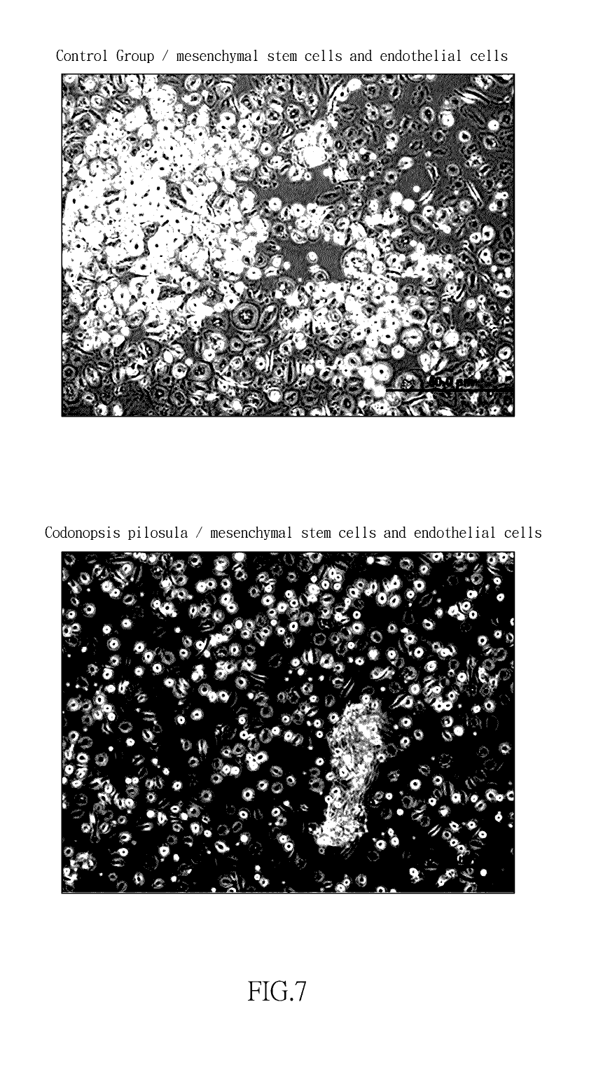

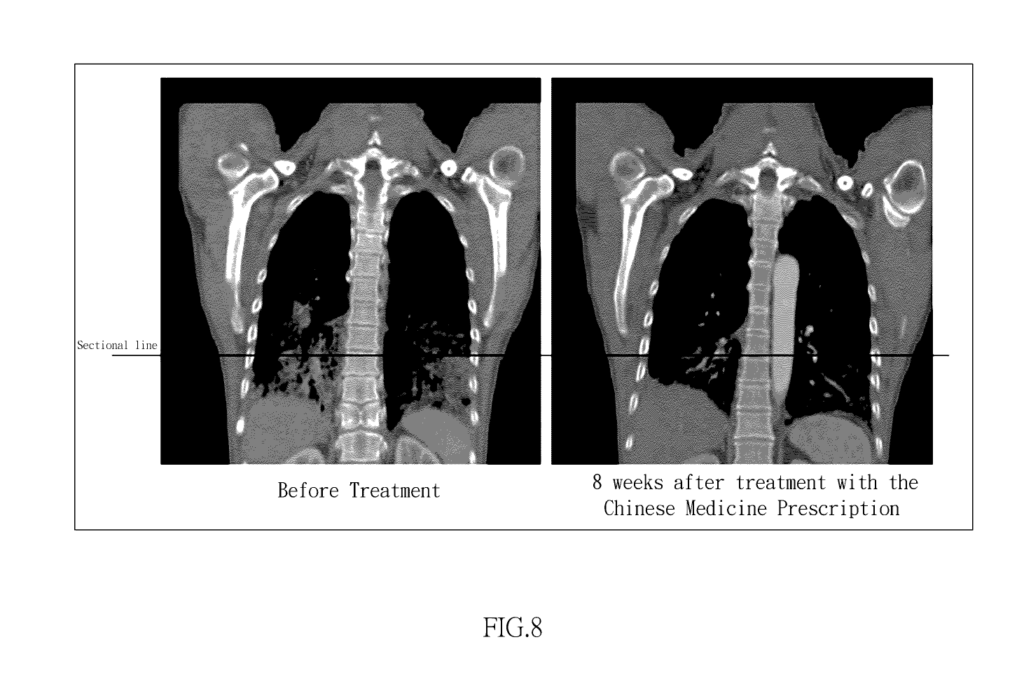

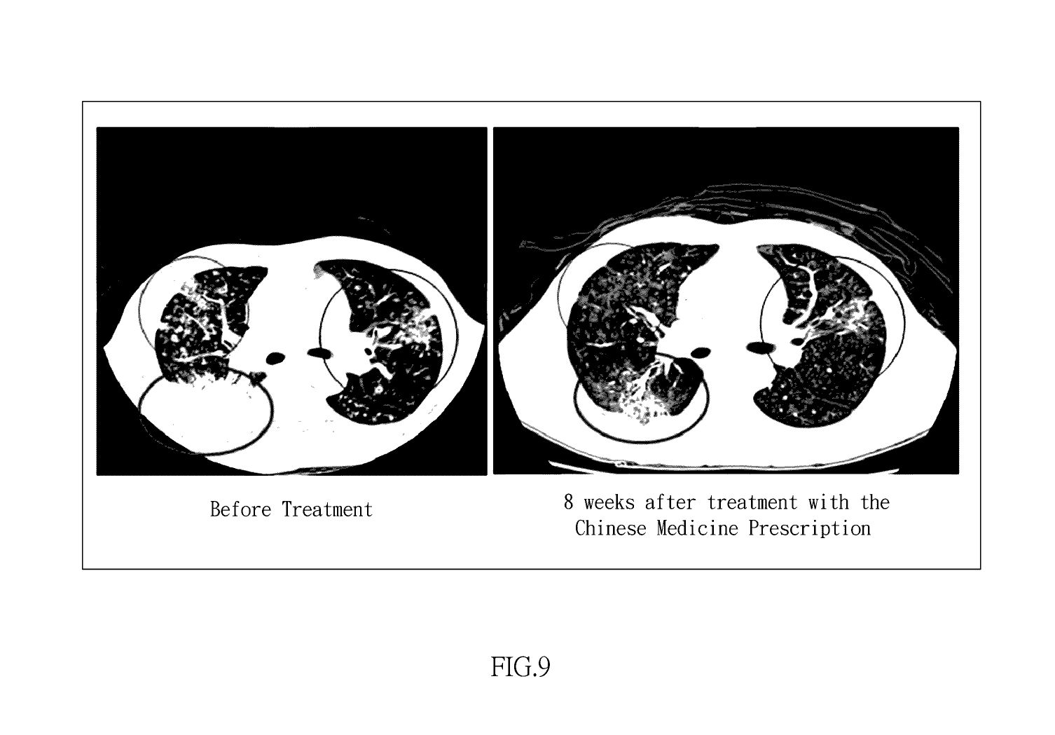

Therefore, this disclosure uses three enzymes including ATAD3A, DRP1 and DDH as the main target proteins in the Chinese medicine prescription, a western blotting assay or an inverted microscope for screening infected lung adenocarcinoma cells, and a computed tomography scan to examine the treatment effect of the Chinese medicine prescription of this disclosure to the lung adenocarcinoma patients. FIG. 6 shows that atractylodes macrocephala and fresh ginger of the Chinese medicine prescription can effectively inhibit the expression of DDH in lung adenocarcinoma cells, and costustoot and Polygala tenuifolia Willd of the Chinese medicine prescription can effectively inhibit the expression of DRP 1 and ATADA3 in the lung adenocarcinomas, wherein costustoot has a preferred inhibiting effect. With reference to FIG. 7 for the mesenchymal stem cells and endothelial cells affected by the expansion and metastasis of the lung adenocarcinoma, the codonopsis pilosula in the Chinese medicine prescription is compared with a control group and screen by an inverted microscope. The area of the flat cells of the affected lung adenocarcinoma can be reduced by more than ⅓ effectively. With reference to FIG. 8 for the treatment effect of the Chinese medicine prescription of this disclosure to lung adenocarcinoma patients examined by the computed tomography, the Chinese medicine prescription capable of treating lung adenocarcinoma in accordance with this disclosure is used for treating a lung adenocarcinoma patient for eight weeks, wherein the patient's lung has white lesions before the treatment. The growth of lung adenocarcinoma cells of the patient's lung is compared with that before taking the treatment, and results show that such white lesions obviously disappears after using the Chinese medicine prescription of this disclosure. In FIG. 9, three major lesion areas marked by circles show that most of the white lesions in the lung adenocarcinoma cells of the patient's lung have disappeared after eight weeks of treatment, particularly the lesion area at the lower left side has a slight trace of pulmonary fibrosis of the patient's lung after the patient takes the treatment.

The aforementioned results show that the Chinese medicine prescription can achieve the expected objectives and trigger lung adenocarcinoma cell autophagy and reduce cancer cell nest growth, expansion and metastasis, and after the Chinese medicine prescription is taken, lung adenocarcinoma cell autophagy occurs to achieve the cancer treatment effect.

What is claimed is:

1. A Chinese medicine prescription capable of treating lung adenocarcinoma, composed of Polygala tenuifolia Willd, atractylodes macrocephala, angelica sinensis, poria pararadicis, astragalus membranaceus, ziziphus jujaba Mill.var.spinosa, codonopsis pilosula, costustoot, radix glycyrrhizae preparata, fresh ginger.

2. The Chinese medicine prescription capable of treating lung adenocarcinoma according to claim 1, wherein the prescription triggers lung adenocarcinoma cell autophagy and inhibits cancer cell nest growth, expansion and metastasis.

3. The Chinese medicine prescription capable of treating lung adenocarcinoma according to claim 2, wherein Polygala tenuifolia Willd, atractylodes macrocephala, fresh ginger, costustoot and codonopsis pilosula are major regulators for inhibiting lung adenocarcinoma cell growth, expansion and metastasis.

4. A composition of a Chinese medicine prescription comprising the Chinese medicine prescription according to claim 1, wherein the composition includes 6 parts by weight of Polygala tenuifolia Willd, 3 parts by weight of atractylodes macrocephala, 3 parts by weight of angelica sinensis, 3 parts by weight of poria pararadicis, 3 parts by weight of astragalus membranaceus, 3 parts by weight of ziziphus jujaba Mill.var.spinosa, 6 parts by weight of codonopsis pilosula, 3 parts by weight of costustoot, 1.5 parts by weight of radix glycyrrhizae preparata, and 1.5 parts by weight of fresh ginger, and the composition of the Chinese medicine prescription is capable of triggering lung adenocarcinoma cell autophagy, reducing cancer cell nest growth, expansion and metastasis, so as to achieve the effect of cancer treatment.

5. The composition of the Chinese medicine prescription according to claim 4, wherein the composition of the Chinese medicine prescription includes a pharmaceutical residue filtered and obtained after a water quenching procedure takes place, so as to obtain a water-quenched medicine soup; and pharmaceutical residue filtered and obtained after processing the water-quenched pharmaceutical residue by a liquor quenching procedure to obtain a liquor-quenched medicine soup; and the water-quenched medicine soup and the liquor-quenched medicine soup are mixed.

6. The composition of the Chinese medicine prescription according to claim 5, wherein the water quenching procedure extracts the herbs by 100 g of water at 60° C. for 20 minutes; and the liquor quenching procedure extracts the herbs by 100 g of 50˜90% alcohol at 60° C. for 15 minutes.
